(19) United States
(12) Patent Application Publication
(10) Pub. No.: US 2007/0179630 A1
(43) Pub. Date: Aug. 2, 2007

(54) Hip stem centralizer cap and method

(75) Inventors: Bart C. Benedict, Warsaw, IN (US); Barish Banerjee, Warsay, IN (US); Ryan T. Ward, Columbia City, IN (US); Singh D. Shankar, Warsaw, IN (US); Timothy L. Downing, Etna Green, IN (US); Scott J. Steffensmeier, Warsaw, IN (US)

Correspondence Address: John F. Hoffman, Esq.;BAKER & DANIELS LLP, Suite 800, 111 East Wayne Street, Fort Wayne, IN, 46802, US

(73) Assignee: Zimmer Technology, Inc.

(21) Appl. No.: 11/345,914

(22) Filed: Feb. 2, 2006

(51) Int. Cl. A61F 2/36; A61F 2/28

(52) U.S. Cl. 623/23.22; 623/23.48

(57) ABSTRACT

A hip stem centralizer is provided for positioning a proximal portion of a femoral hip stem component within an intramedullary canal of a femur. The centralizer includes a cavity and a side wall defining a lateral skirt. The cavity is removably engageable with the proximal portion of the hip stem component to position the lateral skirt between the lateral portion of the hip stem component and the lateral portion of the intramedullary canal.

### FIELD OF THE INVENTION

The present invention relates to total hip arthroplasty. More particularly, the present invention relates to a proximal centralizer for a cemented hip stem and a method for its use.

### BACKGROUND

Total hip arthroplasty is often used to restore function to a diseased or injured hip joint. Positions and directions relative to the hip joint may be described in terms of proximal being nearer the hip joint, distal being further from the hip joint, anterior being nearer the front of the body, posterior being nearer the back of the body, medial being nearer the centerline of the body, and lateral being further from the center line of the body. In total hip arthroplasty (FIGS. 1-2), the articular surfaces of the femur and pelvis are cut away and replaced with prosthetic implant components. In a typical case, the implants include a hip stem component 10, a femoral head component 12, an acetabular component 14, and bone cement 16. The hip stem component includes a stem portion 18 extending down into the intramedullary canal 20 of the femur 22 and a neck portion 24 extending away from the femur 22 to support the femoral head component 12.

The femur 22 is prepared by reaming the intramedullary canal 20 down into the bone along an axis 26 from a proximal position near the hip joint at the upper end of the femur 22 toward a distal position nearer the knee joint at the lower end of the femur 22. The pelvis 28 is prepared by reaming the acetabulum 30. Bone cement 16 is introduced into the prepared intramedullary canal 20 and acetabulum 30 and the prosthetic components are seated in the bone cement 16 so that it hardens around and locks the components in place. Positioning the femoral stem component 10 in the correct orientation within the cement 16 is important for proper biomechanical functioning and long term stability. It is desirable to have a uniform and strong cement mantle 16 proximally around the anterior 34, lateral 36, and posterior 38 portions of the stem component 10. Proper placement further results in appropriate loading of the implants. Femoral components, especially collarless ones, are sometimes placed at the wrong angle in the mediolateral direction. The typical situation is a varus placement in which the angle between the neck 24 and femoral axis 26 is too shallow.

### SUMMARY

The present invention provides a hip stem centralizer for positioning a proximal portion of a femoral hip stem component within an intramedullary canal of a femur.

In one aspect of the invention, the centralizer includes a cavity and a side wall defining a lateral skirt. The cavity is removably engageable with the proximal portion of the hip stem component to position the lateral skirt between the lateral portion of the hip stem component and the lateral portion of the intramedullary canal.

In another aspect of the invention, a combination includes a hip stem component and a hip stem centralizer for positioning the hip stem component within an intramedullary canal of a femur in hip replacement surgery. The centralizer includes a cavity and a side wall defining a lateral skirt. The cavity is removably engageable with the proximal portion of the hip stem component to position the lateral skirt between the lateral portion of the hip stem component and the lateral portion of the intramedullary canal.

In another aspect of the invention, a method of positioning a proximal portion of a femoral hip stem component within an intramedullary canal of a femur includes: introducing bone cement into the intramedullary canal of a femur; inserting a hip stem component into the cement in the intramedullary canal; engaging a hip stem centralizer with a proximal portion of the hip stem component to position a portion of the hip stem centralizer between the lateral portion of the hip stem component and the lateral portion of the intramedullary canal to space the hip stem component a predetermined distance from the lateral portion of the intramedullary canal; and disengaging the hip stem centralizer from the hip stem component once the hip stem component has been positioned.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is an anterior sectional view of a total hip prosthesis implanted in a hip joint;

FIG. 2 is a sectional view taken along line 2-2 of FIG. 1;

FIG. 3 is an exploded perspective view of a hip stem centralizer cap according to the present invention along with an optional inserter and a hip stem prosthesis;

FIG. 4 is an anterior sectional view of the hip stem centralizer cap of FIG. 3 shown in use to position the hip stem prosthesis;

FIG. 5 is a top plan view of the hip stem centralizer cap of FIG. 3 shown in use to position the hip stem prosthesis;

FIG. 6 is an anterior sectional view of the hip stem centralizer cap of FIG. 3 shown in use with the optional inserter of FIG. 3 to position the hip stem prosthesis; and

FIG. 7 is a an anterior sectional view of the hip stem centralizer cap of FIG. 3 shown in use with an alternative optional inserter instrument to position the hip stem prosthesis.

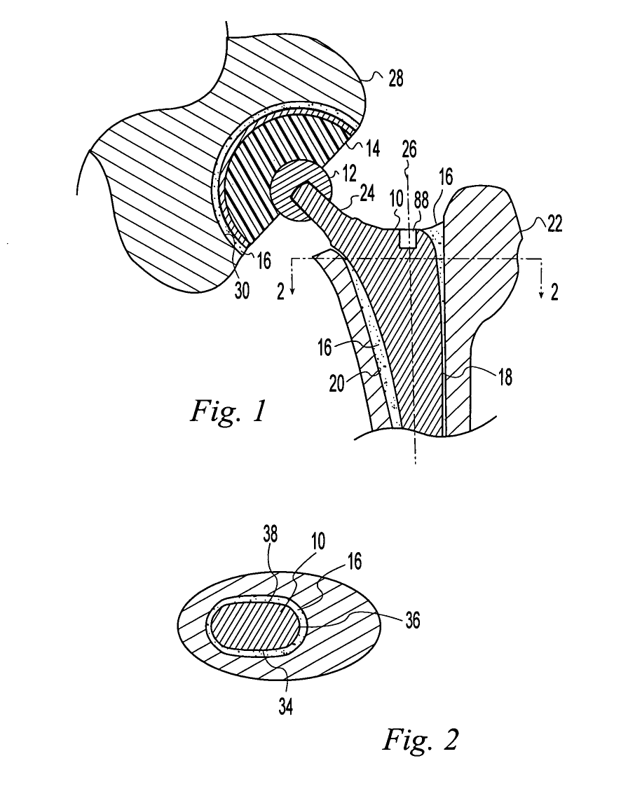

### DESCRIPTION OF THE ILLUSTRATIVE EXAMPLES

Embodiments of a hip stem centralizer cap include a body defining a skirt engageable with the lateral side of a hip stem to space the proximal lateral portion of the hip stem away from the intramedullary canal wall to ensure a minimum cement mantel thickness laterally that is equal to the thickness of the skirt. The cap may include anterior and posterior skirts to space the proximal anterior and posterior aspects of the hip stem away from the intramedullary canal wall also. The anterior, lateral, and posterior skirts may form one continuous skirt. The cap may include an side wall and a cavity formed into the bottom of the body to define the skirt between the side wall and cavity. The cavity may be formed as the negative shape of the proximal portion of the hip stem so that the cavity receives the proximal portion in conforming relationship. The cap may include a handle to facilitate removal of the cap from the hip stem after the hip stem has been positioned.

The cap may be provided in a variety of cavity dimensions to fit different sizes and shapes of hip stem components. The cap may be provided in a variety of skirt thicknesses corresponding to different desired cement mantel thicknesses. The skirt thickness may be provided in any desired thickness.

The cap may include a through bore from the top of the cap to the cavity and along the hip stem axis to receive an inserter through the bore. The inserter may a shaft having a distal end engageable with the proximal end of the hip stem to facilitate axially loading the hip stem to press it into the intramedullary canal. The cap may be slidable axially along the inserter shaft from a first position in which the cap is operably engaged with the hip stem to space the hip stem from the intramedullary canal wall and a second position in which the cap is spaced from the hip stem. The cap may include a ball plunger and the shaft may include an annular groove engageable with the ball plunger to position the cap relative to the annular groove. The shaft may include two annular grooves, one corresponding to each of the axial positions such that the cap snaps into each of the first and second positions. Alternatively, the inserter may include a shoulder engageable with the top of the cap in axial force transmitting relationship to press the cap distally into engagement with the implant.

The cap may be made of metal, plastic, ceramic, and/or any suitable material. The cap may be made of materials that are non-adherent to bone cement to facilitate removal of the cap from the stem as the bone cement cures. For example the cap may be made of a smooth material. In another example, the cap may be made of a naturally lubricious polymer. For example, the cap may be made of polyethylene, polytetrafluroethylene, and/or other naturally lubricious polymers. The cap may be made by machining, molding, and/or other suitable forming process. For example, the cap may be injection molded from a polymer. A molded polymer cap may be sufficiently inexpensive to permit a single use disposable configuration such that cleaning after use is not necessary.

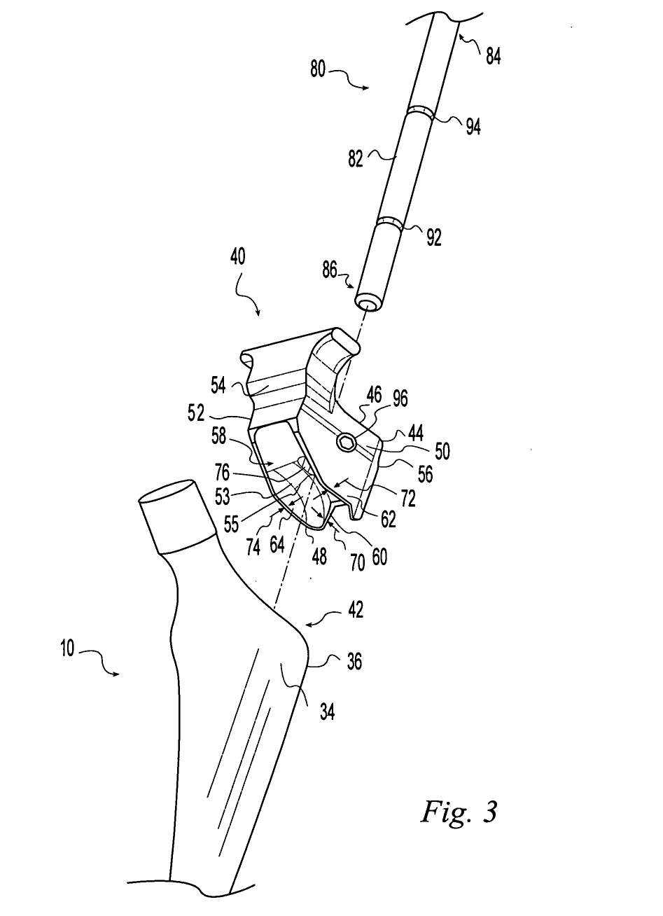

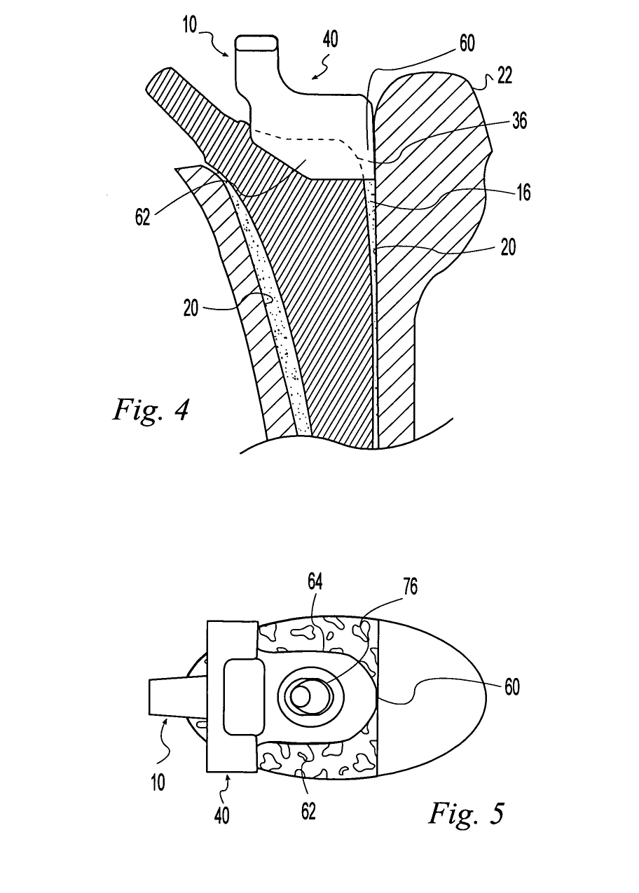

FIG. 3 shows an illustrative hip stem centralizer cap 40 for spacing the proximal portion 42 of the hip stem component 10 from the intramedullary canal wall 20. The cap 40 includes a body 44 having an outer surface including a top 46, a bottom 48, an anterior side 50, a posterior side wall 52, a medial side wall 54, and a lateral side wall 56. A cavity 58 is formed into the bottom 48 and forms a female compliment to the proximal portion 42 of the hip stem component 10. The cavity 58 receives the proximal portion 42 of the hip stem component 10 in conforming relationship. In the illustrative hip stem centralizer cap 40, the cavity 58 includes a medial/lateral curvature 53 matching a corresponding medial/lateral curvature of the proximal portion 42 of the hip stem component 10 and an anterior/posterior curvature 55 matching a corresponding anterior/posterior curvature of the proximal portion 42 of the hip stem component 10. The cavity 58 and side wall of the cap 40 define a thin skirt having a lateral portion 60, an anterior portion 62, and a posterior portion 64. The skirt extends down over the lateral, anterior, and posterior sides 36, 34, 38 of the hip stem component 10. Each portion of the skirt 60, 62, 64 has a thickness 70, 72, 74. The thicknesses of all of the portions may be the same or they may be different. The portions may be any desired thickness. Preferably, the skirt thickness is provided in a range about from 1 mm to about 5 mm. More preferably the skirt has a thickness of approximately 2 mm to 3 mm. In the illustrative embodiment, each portion of the skirt 60, 62, 64 is approximately 2 mm thick. With the cavity 58 engaging the proximal portion 42 of the hip stem component 10 and the outer surface of the cap 40 abutting the intramedullary canal 20 (FIGS. 4-5), the hip stem component is spaced from the intramedullary canal 20 the thickness of the skirt, thus insuring a cement mantel 16 thickness equal to the skirt thickness 70, 72, 74.

A “T”-shaped handle 75 projects proximally and medially from the body 44 and includes ears 77 projecting anteriorly and posteriorly to permit a user to firmly grip the ears 77 and pull the cap 40 from the hip stem component 10 and cement mantel 16.

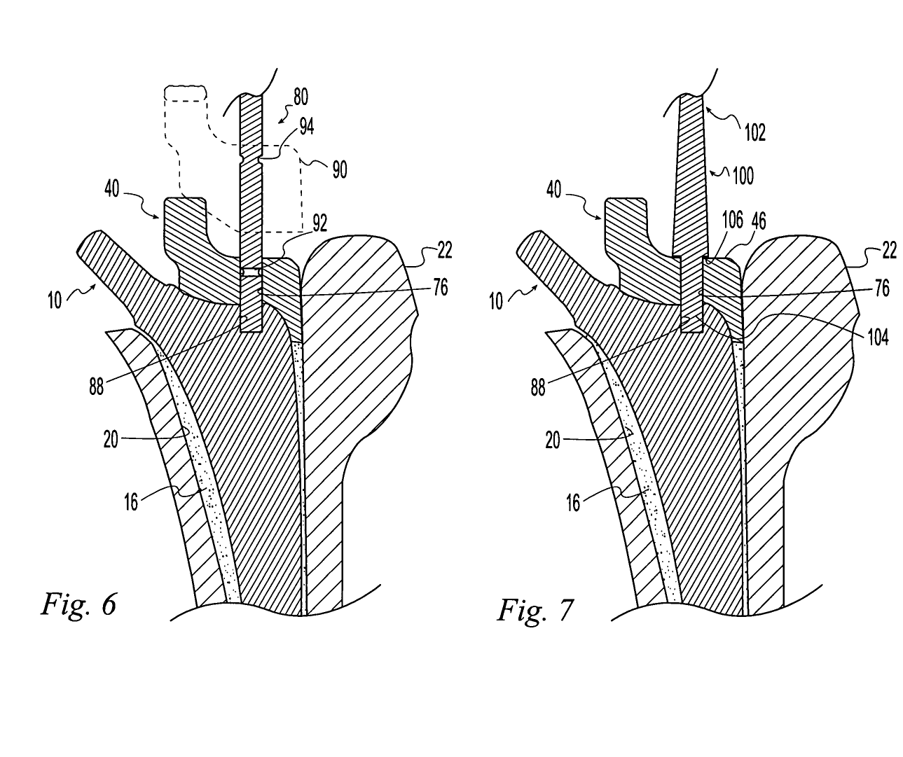

The cap 40 includes a bore 76 from the top 46 through to the cavity 58 along the hip stem axis 26 for receiving an optional stem inserter 80. The stem inserter 80 includes an elongated shaft 82 having a proximal end 84 and a distal end 86 and aligned generally along the hip stem axis 26. The inserter 80 extends through the bore 76 in the cap to engage a hole 88 in the proximal portion 42 of the hip stem component 10 (FIG. 6). The inserter 80 facilitates pushing the hip stem component 10 axially down into the intramedullary canal 20. The cap 40 is slidable along the inserter 80 of FIGS. 3 and 6 from a first position in which the cap 40 is engaged with the proximal portion 42 of the hip stem component 10 and a second position 90 in which the cap 40 is spaced from the hip stem component 10. The inserter shaft 82 includes two annular grooves 92, 94. One of the annular grooves 92 corresponds to the first axial position of the cap 40 and the other annular groove 94 corresponds to the second axial position 90 of the cap 40. The cap 40 includes a ball plunger (not shown) communicating with the bore 76 and retained with a screw 96 in the side of the cap 40. The ball plunger engages the first and second annular grooves 92, 94 to releasably hold the cap 40 in first and second axial positions.

FIG. 7 shows the cap 40 in use with an alternate inserter 100 having a proximal end 102, a distal end 104, and a radially extending shoulder 106 formed adjacent the distal end 104. With the distal end 104 inserted through the bore 76 in the cap and engaged with the hole 88 in the hip stem component 10, the shoulder 106 presses the cap 40 into engagement with the proximal portion 42 of the hip stem component 10.

In use, the intramedullary canal 20 of the femur 22 is reamed to prepare it to receive the hip stem component 10. Bone cement 16 is placed in the intramedullary canal 20 and the hip stem component 10 is pressed into the bone cement 16. The cap 40 is engaged with the proximal portion 42 of the hip stem component 10 and pressed into engagement with the walls of the intramedullary canal 20 anteriorly, posteriorly, and laterally to position the hip stem component 10 at a desired position in the intramedullary canal 20 and to space it from the canal wall to provide a desired cement mantel thickness (FIG. 4) while the cement cures. The cap 40 may remain on the hip stem component 10 until the cement 16 is fully cured. Alternatively, when the bone cement 16 has set sufficiently to retain the hip stem component 10 in the desired position the cap 40 may be removed to help prevent the cap 40 from sticking to the cement 16.

The optional inserter 80 may be used with the cap 40 (FIG. 6) by inserting it through the bore 76 in the cap 40 and into engagement with the hole 88 in the hip stem component 10. The cap 40 is slid down the shaft 82 of the inserter 80 into the first position with the ball plunger snapped into the first annular groove 92. The inserter 80 may be used to initially insert the hip stem component 10 as well as to maintain pressure on the hip stem component 10 while the bone cement 16 hardens. After the bone cement 16 has partially hardened, the cap 40 may optionally be slid back along the shaft 82 to the second position with the ball plunger snapped into the second annular groove 94 to maintain the cap 40 in the second position. Constant pressure may be maintained on the hip stem component 10 while the cap 40 is withdrawn and may be maintained until the cement 16 has fully cured.

The alternative optional inserter 100 (FIG. 7) may be used with the cap 40 by inserting it through the bore 76 in the cap 40 and into engagement with the hole 88 in the hip stem component 10. The shoulder 106 presses the cap 40 firmly against the hip stem component 10. When the cement 16 has hardened, or partially hardened, the inserter 100 and cap 40 are removed.

Although examples of a hip stem centralizer cap and its use have been described and illustrated in detail, it is to be understood that the same is intended by way of illustration and example only and is not to be taken by way of limitation. Accordingly, variations in and modifications to the hip stem centralizer cap and its use will be apparent to those of ordinary skill in the art, and the following claims are intended to cover all such modifications and equivalents.

What is claimed is:

1. A hip stem centralizer for positioning a proximal portion of a femoral hip stem component within an intramedullary canal of a femur, the femoral hip stem component and intramedullary canal each having a proximal portion nearer the hip joint, a distal portion nearer the knee joint, an anterior portion toward the front of the body, a posterior portion toward the rear of the body, and a lateral portion further from the midline of the body, the hip stem centralizer comprising:
a body having a top, a bottom, and a side wall, a cavity being formed into the bottom of the body, the cavity and side wall defining a lateral skirt, the cavity being removably engageable with the proximal portion of the hip stem component to position the lateral skirt between the lateral portion of the hip stem component and the lateral portion of the intramedullary canal.

2. The hip stem centralizer of claim 1 wherein the cavity and side wall further define an anterior skirt and a posterior skirt, the cavity being removably engageable with the proximal portion of the hip stem component to position the anterior, lateral, and posterior skirts between the anterior, lateral, and posterior portions of the hip stem component and intramedullary canal.

3. The hip stem centralizer of claim 2 wherein the anterior, lateral, and posterior skirts form one continuous skirt.

4. The hip stem centralizer of claim 1 further comprising a handle extending proximally from the body.

5. The hip stem centralizer of claim 4 wherein the handle includes a pair of ears extending anteriorly and posteriorly.

6. The hip stem centralizer of claim 1 further comprising a plurality of similarly formed bodies, the bodies varying in cavity dimension.

7. The hip stem centralizer of claim 1 further comprising a plurality of similarly formed bodies, each body defining a medial/lateral skirt thickness, each body having a different skirt thickness.

8. The hip stem centralizer of claim 1 wherein the body includes a through bore from the top distally into the cavity, the hip stem centralizer further comprising an inserter comprising a longitudinal shaft having a proximal end, a distal end, and a axis extending therebetween, the body being mounted on the shaft with the shaft engaging the through bore.

9. The hip stem centralizer of claim 8 wherein the body is slidable axially along the inserter shaft from a first position in which the cavity is relatively nearer the distal end of the shaft and a second position relatively further from the distal end of the shaft.

10. The hip stem centralizer of claim 8 wherein the body includes a plunger biased into the bore and the inserter shaft includes a first annular groove engageable with the plunger in the first position and a second annular groove engageable with the plunger in the second position.

11. The hip stem centralizer of claim 8 wherein the shaft includes a shoulder extending radially outwardly from the shaft axis, the shoulder abutting the top of the body in axial force transmitting relationship.

12. A combination of a hip stem component and a hip stem centralizer for positioning the hip stem component within an intramedullary canal of a femur in hip replacement surgery, the intramedullary canal having a proximal portion nearer the hip joint, a distal portion nearer the knee joint, an anterior portion toward the front of the body, a posterior portion toward the rear of the body, and a lateral portion further from the midline of the body, the combination comprising:
a hip stem component comprising a proximal portion having an anterior side, a lateral side, and a posterior side, the proximal portion defining a proximal portion shape; and
a hip stem centralizer comprising a body having a top, a bottom, and a side wall, a cavity being formed into the bottom of the body, the cavity conforming to the proximal portion shape, the cavity and side wall defining a lateral skirt, the cavity being removably engageable with the proximal portion of the hip stem component to position the lateral skirt between the lateral portion of the hip stem component and the lateral portion of the intramedullary canal.

13. The combination of 12 wherein the cavity and side wall further define an anterior skirt and a posterior skirt, the cavity being removably engageable with the proximal portion of the hip stem component to position the anterior, lateral, and posterior skirts between the anterior, lateral, and posterior portions of the hip stem component and intramedullary canal.

14. The combination of claim 12 further comprising a handle extending proximally from the body.

15. The combination of claim 12 wherein the body includes a through bore from the top distally into the cavity, the hip stem centralizer further comprising an inserter comprising a longitudinal shaft having a proximal end, a distal end, and a axis extending therebetween, the body being mounted on the shaft with the shaft engaging the through bore.

16. The combination of claim 15 wherein the body is slidable axially along the inserter shaft from a first position in which the body is adjacent to the hip stem component with the cavity engaged with the proximal portion of the hip stem component and a second position in which the body is spaced from the hip stem component.

17. The combination of claim 15 wherein the shaft includes a shoulder extending radially outwardly from the shaft axis, the shoulder abutting the top of the body in axial force transmitting relationship such that a distal axial force presses the hip stem centralizer distally into engagement with the proximal portion of the hip stem component.

18. A method of positioning a proximal portion of a femoral hip stem component within an intramedullary canal of a femur, the method comprising:
introducing bone cement into the intramedullary canal of a femur;
inserting a hip stem component into the cement in the intramedullary canal;
engaging a hip stem centralizer with a proximal portion of the hip stem component to position a portion of the hip stem centralizer between the lateral portion of the hip stem component and the lateral portion of the intramedullary canal to space the hip stem component a predetermined distance from the lateral portion of the intramedullary canal; and
disengaging the hip stem centralizer from the hip stem component once the hip stem component has been positioned.

19. The method of claim 18 wherein engaging a hip stem centralizer with a proximal portion of the hip stem component further comprises positioning a portion of the hip stem centralizer between the anterior and posterior portions of the hip stem component and the intramedullary canal to space the hip stem component a predetermined distance from the anterior and posterior portion of the intramedullary canal.

20. The method of claim 18 further comprising waiting for the bone cement to partially harden before disengaging the hip stem centralizer.

21. The method of claim 18 further comprising waiting for the bone cement to fully harden before disengaging the hip stem centralizer.

22. The method of claim 18 wherein the hip stem centralizer comprises a shaft and a body engageable with the proximal portion of the hip stem component, the body being slidable along the shaft from a first position in which the body is engaged with the hip stem component and a second position in which the body is spaced from the hip stem component, the method further comprising applying a distal axial force to the hip stem component with the shaft; and wherein disengaging the hip stem centralizer comprise sliding the body from the first to the second position.

23. The method of claim 22 further comprising waiting for the bone cement to at least partially harden before sliding the body from the first to the second position.

24. The method of claim 18 wherein the hip stem centralizer comprises a shaft and a body engageable with the proximal portion of the hip stem component and wherein engaging a hip stem centralizer with a proximal portion of the hip stem component comprises applying a distal axial force to the body to press it into engagement with the proximal portion of the hip stem component.
